(19) United States
(12) Patent Application Publication
(10) Pub. No.: US 2018/0095042 A1
(43) Pub. Date: Apr. 5, 2018

(54) Method For Detecting Treated Wood

(71) Applicant: University of South Carolina, Columbia, SC (US)

(72) Inventors: John L. Ferry, Columbia, SC (US); Michael C. Bagley, Columbia, SC (US)

(21) Appl. No.: 15/719,790

(22) Filed: Sep. 29, 2017

Related U.S. Application Data
(60) Provisional application No. 62/401,998, filed on Sep. 30, 2016.

(51) Int. Cl. G01N 21/78; G01N 33/46

(52) U.S. Cl. CPC G01N 21/78; G01N 33/46

(57) ABSTRACT

The present disclosure is directed to a method for detecting whether a wood has been treated with a metal. The method comprises a step of applying a solution containing a color change agent and an antioxidant to the wood. The present disclosure also provides a kit for determining whether a wood has been treated with a metal. The kit comprises a compartment containing a solution containing a color change agent and an antioxidant and an applicator for applying the solution to the wood. The present disclosure is also directed to a solution for detecting treated wood wherein the solution comprises a color change agent and an antioxidant.

### BACKGROUND

Wood used for outdoor construction has been historically treated with mixtures of metals and/or metal salts to inhibit decay. Early versions of this technology relied on the application of mixtures of copper, chromium, and arsenic salts to wood surfaces at high pressures to inoculate the wood against microbes. Today, the bulk of treated wood is produced by treating wood with high pressure solutions of copper salts of antimicrobial amines. However, this wood, when recycled, is generally not suitable for use in certain applications and in certain environments. For instance, this wood may not be suitable for use inside homes, as composting, as a source of paper products, or as burning fuel because of its high copper content.

Typically, tests are performed that allow for the determination of whether the wood has been treated. In some instances, waste management is encouraged to pick up the wood during collection and determine whether it has been treated. However, using current methods, the time period required is longer than desired. In addition to this, current solutions have a tendency to degrade in a short period of time and thus have a minimal shelf life.

As a result, there is a need for providing an efficient method for determining whether wood has been treated with metals, such as copper. Such a method can be used to determine whether the wood is suitable for re-use for a certain application and/or whether additional treatment is required. In addition, there is a need for providing a stable solution that allows for the determination of whether wood has been treated with metals, such as copper.

### SUMMARY

In general, according to one embodiment, the present disclosure is directed to a method for detecting whether a wood has been treated with a metal. The method comprises a step of applying a solution containing color change agent and an antioxidant to the wood.

In general, according to another embodiment, the present disclosure is directed to a solution for detecting treated wood. The solution comprises a color change agent and an antioxidant.

In general, according to another embodiment, the present disclosure provides a kit for determining whether a wood has been treated with a metal. The kit comprises a compartment containing a solution containing a color change agent and an antioxidant and an applicator for applying the solution to the wood.

Other features and aspects of the present disclosure are discussed in greater detail below.

### DETAILED DESCRIPTION

Reference now will be made in detail to the embodiments of the invention, one or more examples of which are set forth below. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment. Thus, it is intended that the present invention cover such modifications and variations.

In general, the present invention is directed to a method of detecting whether wood has been treated. In particular, the present invention is directed to a method of detecting whether wood has been treated with a metal.

As used herein, metals are defined to include metals as well as their corresponding metal salts (e.g., copper and copper salts, etc.). According to the present invention, the metal may include copper. In one particular embodiment, the metal includes copper. In such embodiment, the metal may be copper (II) and/or copper (I). In one embodiment, the metal may be a reduced metal. For instance, the metal may be copper (I) reduced from copper (II), so that copper (I) is a reduced metal. In one embodiment, the metal may be copper (II).

According to one embodiment of the present disclosure, the method comprises a step of applying a color change agent to the wood. In general, the color change agent may be any compound capable of reacting with a metal and/or forming a complex with a metal, such as a reduced metal, wherein such reaction and/or complexation results in a color change. Such reaction may be the result of the formation of a metal-color change agent complex. In this regard, the color change agent may be a colorimetric molecule.

In one embodiment, the color change agent may be 5-{[(1E)-3-carboxylato-5-methyl-4-oxocyclohexa-2,5-dien-1-ylidene](2,6-dichloro-3-sulfonatophenyl)methyl}-2-hydroxy-3-methylbenzoate or a salt thereof. For instance, the salt may be an alkali metal salt. For instance, the alkali metal salt may be a sodium salt, such as a monosodium salt (e.g., monosodium salt hydrate), disodium salt, and/or trisodium salt. For instance, in one embodiment, the color change agent may be a trisodium salt, such as trisodium 5-{[(1E)-3-carboxylato-5-methyl-4-oxocyclohexa-2,5-dien-1-ylidene](2,6-dichloro-3-sulfonatophenyl)methyl }-2-hydroxy-3-methylbenzoate. Such color change agent may also be known in the art as chromazurol S.

In one embodiment, the color change agent may be a salt of the following compound:

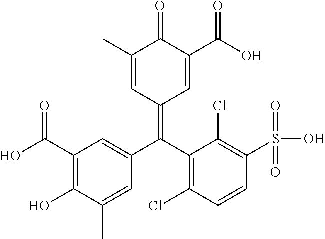 [SMILES: CC1=C/C(=C(\c2cc(C)c(O)c(C(=O)O)c2)c2c(Cl)ccc(S(=O)(=O)O)c2Cl)C=C(C(=O)O)C1=O]

For instance, in one embodiment, the color change agent may be a monovalent salt of the aforementioned compound. In another embodiment, the color change agent may be a divalent salt of the aforementioned compound. In a further embodiment, the color change agent may be a trivalent salt of the aforementioned compound.

In one particular embodiment, the color change agent may be a trivalent salt of the aforementioned compound, such as a trisodium salt. The structure of trisodium 5-[(E)-(3-carboxylato-5-methyl-4-oxocyclohexa-2,5-dien-1-ylidene)-(2,6-dichloro-3-sulfophenyl)methyl]-3-methyl-2-oxidobenzoate is as follows:

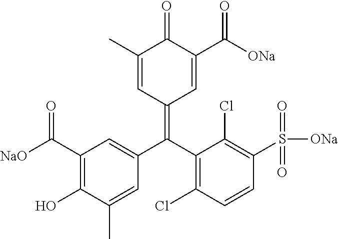

However, it should be understood that the compound may undergo partial protonation such that the compound is a monovalent compound or a divalent compound thus resulting in a monovalent salt, such as a monosodium salt, or a divalent salt, such as disodium salt, respectively.

Thus, when the color change agent is chromazurol S as defined herein and copper is being detected, a copper-chromazurol S complex, such as a copper (II)-chromazurol S complex and/or a copper (I) chromazurol S complex, may be formed. In one embodiment, the complex may be a copper (II)-chromazurol S complex. In another embodiment, the complex may be a copper (II)-chromazurol S complex.

In this regard, the color change agent may form a complex with the copper present in the wood. While chromazurol S is mentioned above, it should be understood that the complex may be formed with any of the aforementioned compounds in any of the aforementioned protonation states. For instance, the complex may be formed with the monovalent compound, such as the monovalent salt, and/or the divalent compound, such as the divalent salt.

The color change agent may be a solid. When in the form of a solid, such as a powder, it may be delivered via a liquid. For instance, the color change agent may be dissolved and/or suspended within a liquid such that it is delivered via a liquid phase. In one embodiment, the color change agent is dissolved and/or suspended in a liquid and delivered as a solution. For instance, the liquid (or solvent) may be water, an organic solvent, or a mixture thereof. The organic solvent may include any types of organic solvents known in the art. For instance, the organic solvents include, but are not limited to, alcohols (e.g., methanol, ethanol, isopropanol, etc.), glycols (e.g., ethylene glycol such as monoethylene glycol, propylene glycol, etc.).

In one embodiment, the color change agent may be chromazurol S as identified above and it may be dissolved or delivered via water. In another embodiment, the color change agent may be chromazurol S as identified above and it may be dissolved or delivered via a mixture of water and an organic solvent. For instance, the organic solvent may be an alcohol, a glycol, or a combination thereof. In one embodiment, the organic solvent includes both an alcohol and a glycol. For instance, the organic solvent may include both ethanol and ethylene glycol, such as monoethylene glycol.

In one embodiment, solutions or suspensions/dispersions disclosed herein may be directly applied to the wood, such as to a surface of the wood. In another embodiment, the color change agent may be in the form of an ink or a spray and directly marked on or applied to the wood. In another embodiment, the color change agent may be in the form of a solid or a powder and directly applied to the wood. In such instance, the wood may be wet or damp and capable of dissolving/delivering the color change agent and/or facilitating a reaction between or complexation of the color change agent and the metal.

In one embodiment, the color change agent has a high purity. For instance, the color change agent may have a purity of at least 80%, such as at least 85%, such as at least 90%, such as at least 95%, such as at least 98%, such as at least 99%, such as at least 99.5%, such as at least 99.9%, such as at least 99.99%. The purity can be determined using any method generally employed in the art. Without intending to be limited by theory, it is believed that having a color change agent with such purity may contribute to the stability of such agent.

In one embodiment, an antioxidant may also be employed. In general, without intending to be limited by theory, the antioxidant may serve as a preservative for the color change agent. For instance, the antioxidant may prevent the color change agent from becoming unstable due to the presence of contaminants, such as iron (III). The antioxidant may be any compound capable of serving as a preservative for the color change agent and protect the color change agent from oxidants, for example by reducing them.

In general, a variety of antioxidants may be suitable. For example, suitable classes of antioxidant compounds include hydrazines, phosphites (e.g., alkylated aryl phosphites, sterically hindered aryl phosphites, aliphatic spirocyclic phosphites, trialkyl phosphites, mixed alkyl/aryl phosphites, etc.), sterically hindered bisphosphonites, sulfites, hydrosulfides, hydroxyphenyl propionates, hydroxy benzyls, aromatic amines, hindered amines, hydroquinones, sterically hindered phenyl spirocyclics, sterically hindered bisphosphonites, thiosemicarbazides, alkylidene bisphenols, alkyl phenols, thioethers, and mixtures thereof.

In one embodiment, the antioxidant is a phenolic compound. For instance, the phenolic antioxidant may include, for example, 4,4′-thiobis-6-t-butylmethylphenol, butylated hydroxyanisole (mixture of 2-t-butyl-4-methoxyphenol and 3-t-butyl-4-methoxyphenol), p-octyl phenol, mono (di or tri)-(α-methylbenzyl) phenol, 2,6-di-t-butyl-p-cresol (BHT) and pentaerythrithyl tetrakis [3-(3,5-di-t-butyl-4-hydroxyphenyl)]propionate; an amine-based antioxidant such as N,N′-di-2-naphthyl-p-phenylene diamine; a hydroquinoline-based antioxidant such as 2,5-di(t-amyl)hydroquinoline; a sulfur-based antioxidant such as dilauryl thiodipropionate; a phosphorus-based antioxidant such as triphenyl phosphite and 3,5-di-tert-butyl-hydroxycinnamate (known as IRGANOX 1076, commercially available from Ciba Geigy).

In one particular embodiment, the antioxidant may be a hindered phenolic compound having the formula:

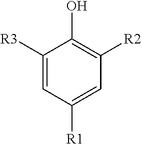

wherein

R1, R2, and R3 are independently selected from hydrogen, halogen, a C(₁₋₁₂) alkoxy, or a C(₁₋₁₂) alkyl group.

In one embodiment, R1 is a C(₁₋₁₂) alkoxy or a C(₁₋₁₂) alkyl group, such as a C(₁₋₉) alkoxy, or a C(₁₋₉) alkyl group, such as a C(₁₋₅) alkoxy, or a C(₁₋₅) alkyl group, such as a C(₁₋₃) alkoxy, or a C(₁₋₃) alkyl group, such as a C(₁₋₂) alkoxy, or a C(₁₋₂) alkyl group, such as a methoxy or a methyl group. In particular embodiment, R1 is methyl. In another particular embodiment, R1 is methoxy.

In one embodiment, R2 is a hydrogen, a C(₁₋₁₂) alkoxy, or a C(₁₋₁₂) alkyl group, such as a hydrogen or a C(₁₋₉) alkyl group, such as a hydrogen or a C(₁₋₆) alkyl group, such as a hydrogen or a C(₂₋₆) alkyl group, such as a hydrogen or a C(₃₋₅) alkyl group, such as a hydrogen or a butyl group, such as a hydrogen or a tert-butyl group. In one particular embodiment, R2 is hydrogen. In another particular embodiment, R2 is a butyl group, such as a tert-butyl group.

In one embodiment, R3 is a hydrogen, a C(₁₋₁₂) alkoxy, or a C(₁₋₁₂) alkyl group. In one particular embodiment, R3 is a C(₁₋₁₂) alkyl group, such as a C(₁₋₉) alkyl group, such as a C(₁₋₆) alkyl group, such as a C(₂₋₆) alkyl group, such as a C(₃₋₅) alkyl group, such as a butyl group, such as a tert-butyl group. In another particular embodiment, R3 is a butyl group, such as a tert-butyl group.

In one embodiment, R1 is methyl and R2 and R3 are both tert-butyl (butylated hydroxytoluene (BHT)). In another embodiment, R1 is methoxy, R2 is hydrogen, and R2 is tert-butyl (butylated hydroxy anisole (BHA)). In one particular embodiment, the antioxidant, such as the phenolic antioxidant, such as the hindered phenolic antioxidant, comprises butylated hydroxytoluene.

In one embodiment, the antioxidant is a toluene or a derivative thereof. For instance, the toluene may be a hydroxytoluene. In particular, the toluene may be an alkylated hydroxytoluene. The alkyl groups may be C1-C5 groups, such as butyl groups. In this regard, the antioxidant may be a butylated hydroxytoluene.

Other examples of antioxidants include, but are not limited to tocopherols, polyphenols such as flavonoids and other naturally occurring polyphenols.

Typically, conventional solutions containing the color change agent degrade within 1 to 2 weeks. Providing the antioxidant in the manner as disclosed herein can provide a stable solution. For instance, the solution can be stable for at least 2 weeks, such as at least 3 weeks, such as at least 4 weeks, such as at least 6 weeks, such as at least 8 weeks, such as at least 12 weeks, such as at least 16 weeks, such as at least 20 weeks, such as at least 24 weeks. The stability is determined based on the degradation of the color change agent.

In one embodiment, the composition may also contain a buffer. The buffer may be an acidic buffer. For instance, the buffer may be employed to provide a pH of from 3 to 7, such as from 5 to 7, such as from 5 to less than 7.

In general, the buffer may not complex with the copper. In this regard, the buffer may include, but is not limited to, an acetate buffer, a GTA buffer, an oxalic acid buffer, a succinate buffer, and others. In one particular embodiment, the buffer includes an acetate buffer. In one particular embodiment, the acetate buffer comprises an alkali metal acetate buffer. In one particular embodiment, the acetate buffer comprises a sodium acetate buffer.

In the solution, the concentration of the color change agent can be 1 micromolar or greater, such as 100 micromolar or greater, such as 150 micromolar or greater, such as 250 micromolar or greater, such as 500 micromolar or greater, such as 700 micromolar or greater, such as 900 micromolar or greater, such as 1 millimolar or greater, such as 10 millimolar or greater, such as 50 millimolar or greater, such as 100 millimolar or greater. In the solution, the concentration of the color change agent can be 500 millimolar or less, such as 100 millimolar or less, such as 10 millimolar or less, such as 1 millimolar or less, such as 900 micromolar or less, such as 700 micromolar or less, such as 500 micromolar or less, such as 250 micromolar or less, such as 150 micromolar or less, such as 100 micromolar or less, such as 50 micromolar or less.

In the solution, the concentration of the antioxidant can be 1 micromolar or greater, such as 100 micromolar or greater, such as 150 micromolar or greater, such as 250 micromolar or greater, such as 500 micromolar or greater, such as 700 micromolar or greater, such as 900 micromolar or greater, such as 1 millimolar or greater, such as 10 millimolar or greater, such as 50 millimolar or greater, such as 100 millimolar or greater. In the solution, the concentration of the antioxidant can be 500 millimolar or less, such as 100 millimolar or less, such as 10 millimolar or less, such as 1 millimolar or less, such as 900 micromolar or less, such as 700 micromolar or less, such as 500 micromolar or less, such as 250 micromolar or less, such as 150 micromolar or less, such as 100 micromolar or less, such as 50 micromolar or less.

In the solution, the molar ratio of the color change agent to the antioxidant can be 0.00001 or greater, such as 0.00002 or greater, such as 0.0001 or greater, such as 0.001 or greater, such as 0.01 or greater, such as 0.1 or greater, such as 0.25 or greater, such as 0.5 or greater, such as 0.7 or greater, such as 0.9 or greater. In the solution, the molar ratio of the color change agent to the antioxidant can be 5000 or less, such as 4000 or less, such as 3000 or less, such as 2000 or less, such as 1000 or less, such as 500 or less, such as 400 or less, such as 250 or less, such as 150 or less, such as 100 or less, such as 75 or less, such as 50 or less, such as 25 or less, such as 10 or less, such as 5 or less, such as 2 or less, such as 1.5 or less, such as 1.25 or less, such as 1.1 or less.

By providing a solution having the concentrations and/or ratios as defined herein, the kinetics of the complexation can occur in an efficient and/or quick manner. For instance, when the copper is present in a low amount, having a sufficient amount of color change agent can allow for an efficient and/or quick complexation.

The method employed herein is capable of detecting whether the wood has been treated, such as with a metal. For instance, when forming a metal-color change agent complex, a color change may be detected. In one embodiment, when the metal comprises copper and the color change agent comprises 5-{[(1E)-3-carboxylato-5-methyl-4-oxocyclohexa-2,5-dien-1-ylidene](2,6-dichloro-3-sulfonatophenyl)methyl}-2-hydroxy-3-methylbenzoate or a salt thereof, such as trisodium 5-{[(1E)-3-carboxylato-5-methyl-4-oxocyclohexa-2,5-dien-1-ylidene](2,6-dichloro-3-sulfonatophenyl)methyl}-2-hydroxy-3-methylbenzoate, the formation of a copper-5-{[(1E)-3-carboxylato-5-methyl-4-oxocyclohexa-2,5-dien-1-ylidene](2,6-dichloro-3-sulfonatophenyl)methyl}-2-hydroxy-3-methylbenzoate complex may yield a color, such as a blue or green color. The presence of such complex can be determined by measuring the optical absorbance of a solution containing such complex. For instance, in one embodiment, if the complex is present, a maximum optical absorbance of between 400 nm and 600 nm, such as from 500 nm to 600 nm, such as from 550 nm to 600 nm, may be observed.

The color change may occur after a sufficient period of time to allow for such reaction/complexation. For instance, the color change may occur after a sufficient period of time for allowing a reaction or complexation to occur between the color change agent and the metal, such as the reduced metal.

In one embodiment, the color change may be observed within 1 minute, such as within 2 minutes, such as within 5 minutes, such as within 10 minutes, such as within 15 minutes, such as within 30 minutes, such as within 1 hour, such as within 2 hours, such as within 5 hours.

In one embodiment, visual inspection may be used to determine the presence of a metal by a change in color. For instance, the color change may be an observable color change. In one embodiment, the presence of a color change may indicate the presence of copper, such that the wood had been treated with copper.

In general, according to the method disclosed herein, the wood is not particularly limited. For instance, the wood may be a hardwood or a softwood. The softwoods may comprise pine, juniper, redwood, yew, spruce, fir, Douglas fir, cedar, yellow cedar, cyprus, larch, hemlock, etc. The hardwoods may comprise aspen, alder, balsa, mahogany, teak, poplar wood, cottonwood, eucalyptus, maple, birch, beech, oak, hickory, walnut, black locust, etc.

In addition, it should be understood that the wood may be in any shape or form. For instance, the wood may be, but is not limited to, shredded wood, wood chips, intact wood, sawdust, etc. The wood may be, but is not limited to, lumber, plywood, oriented strandboard, and paper.

The wood may also include any composite that contains at least 10 wt. % wood, such as at least 25 wt. % wood, such as at least 50 wt. % wood, such as at least 75 wt. % wood, such as at least 90 wt. % wood, such as at least 95 wt. % wood.

When applying a color change agent and optionally an antioxidant, it should be understood that such applications may be on any part of the wood. For instance, such application may be on an exterior surface of the wood. Alternatively or in addition, such application may be on an interior surface of the wood.

The present disclosure also relates to a composition containing the aforementioned color change agent and antioxidant. The composition may also contain the aforementioned solvent or liquid. Also, the composition may contain the color change agent and the antioxidant in the aforementioned concentrations and/or ratios.

The present disclosure also relates to an apparatus or kit for testing and/or determining whether wood has been treated with a metal. In one embodiment, the apparatus/kit may include a handheld device. Such a device may enable an individual (e.g., consumer, industrial worker, etc.) to differentiate between treated and untreated wood on-site without significant time delays.

The present disclosure also relates to an apparatus for testing or determining whether wood has been treated with a metal. In one embodiment, the apparatus may be a handheld device. Such a device may enable an individual (e.g., consumer, industrial worker, etc.) to differentiate between treated and untreated wood on-site without significant time delays.

The apparatus/kit may include at least one compartment containing the color change agent. In one embodiment, the apparatus/kit may include at least one compartment containing both the color change agent and the antioxidant. In another embodiment, the apparatus/kit may include at least two compartments, wherein a first compartment contains the color change agent while a second compartment contains the antioxidant. In one embodiment, when provided in separate compartments, the antioxidant, when necessary, and the color change agent may be combined prior to application. In another embodiment, when provided in separate compartments, the antioxidant, when necessary, and the color change may not be combined after application. For instance, the antioxidant, when necessary, and the color change agent may be dispersed simultaneously from the apparatus through separate ejection points. Alternatively, the antioxidant, when necessary, and the color change agent may be dispersed at separate times through their corresponding ejection points. It should be understood, however, that the antioxidant, when necessary, and color change agent can be ejected from the apparatus using any method known in the art.

In addition, in the apparatus/kit, the color change agent may be present in the form of a solid, such as a powder, or may be present as a solution. Likewise, the antioxidant, in the apparatus, may be in the form a solution.

In one embodiment, the compartment(s) may be provided with a cartridge or cartridges, such that the cartridge is removable. For instance, when a cartridge and/or compartment are empty, it can be removed from the apparatus/kit and replaced by inserting another cartridge. In this regard, the apparatus/kit may be a refillable apparatus/kit. For instance, the apparatus/kit may be a pen or pen-like device that is refillable with another compartment or cartridge. Alternatively, the compartment or cartridge may remain within the apparatus/kit and may be capable of being refilled with the appropriate component (i.e., antioxidant, color change agent, etc.). In this regard, rather than removing the compartment or cartridge, it can simply be refilled with the appropriate component.

The apparatus/kit may also include a means for bringing the color change agent and/or antioxidant into contact with the wood and/or metal of the wood. Such applicator may include, but are not limited to, a brush, a pipet, a wipe, a marking pen, a needle, etc. The apparatus/kit may also include a means for sampling (e.g., abrasive, knife, etc.) the wood and written instructions for performing the test.

The method disclosed herein can be used to detect the metals and limit human contact and interaction with toxic materials. Thus, once treated wood is identified, it can be decontaminated and recycled using various processes as known in the art. One example of a process for recycling treated wood is disclosed in U.S. Pat. No. 6,035,791 to Hery et al. Thus, the method disclosed herein can make recycling more efficient, in particular by allowing places such as recycling cents to immediately determine whether wood is suitable for re-use or requires additional treatment in order to decrease the amount of any chemicals or materials that would be harmful for the environment.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

1. A method for detecting whether a wood has been treated with a metal, the method comprising applying a solution containing a color change agent and an antioxidant to the wood.

2. The method according to claim 1, wherein the color change agent comprises 5-{[(1E)-3-carboxylato-5-methyl-4-oxocyclohexa-2,5-dien-1-ylidene](2,6-dichloro-3-sulfonatophenyl)methyl}-2-hydroxy-3-methylbenzoate or a salt thereof.

3. The method according to claim 2, wherein the color change agent comprises an alkali metal salt.

4. The method according to claim 2, wherein the color change agent comprises a trisodium salt.

5. The method according to claim 1, wherein the antioxidant comprises a phenolic compound.

6. The method according to claim 5, wherein the phenolic compound comprises a compound having the formula 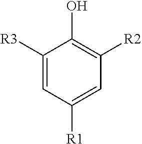
wherein
R1, R2, and R3 are independently selected from hydrogen, halogen, a C(₁₋₁₂) alkoxy, or a C(₁₋₁₂) alkyl group.

7. The method according to claim 1, wherein the antioxidant comprises a toluene or a derivative thereof.

8. The method according to claim 7, wherein the antioxidant comprises a butylated hydroxytoluene.

9. The method according to claim 1, wherein the metal comprises a copper.

10. The method according to claim 1, wherein the solution further comprises a buffer.

11. The method according to claim 1, wherein the concentration of the color change agent is from 1 micromolar to 1 millimolar, wherein the concentration of the antioxidant is from 1 micromolar to millimolar, or a combination thereof.

12. The method according to claim 1, wherein the molar ratio of the color change agent to the antioxidant is from 0.00001 to 5000.

13. The method according to claim 1, wherein the solution is stable for at least 3 weeks.

14. A solution for detecting treated wood, the solution comprising:
a color change agent and an antioxidant.

15. The solution according to claim 14, wherein the color change agent comprises 5-{[(1E)-3-carboxylato-5-methyl-4-oxocyclohexa-2,5-dien-1 -ylidene](2,6-dichloro-3-sulfonatophenyl)methyl}-2-hydroxy-3-methylbenzoate or a salt thereof and the antioxidant comprises a phenolic compound.

16. The solution according to claim 14, wherein the color change agent comprises an alkali metal salt of 5-{[(1E)-3-carboxylato-5-methyl-4-oxocyclohexa-2,5-dien-1-ylidene](2,6-dichloro-3-sulfonatophenyl)methyl}-2-hydroxy-3-methylbenzoate.

17. The solution according to claim 14, wherein the antioxidant comprises a butylated hydroxytoluene.

18. The solution according to claim 14, wherein the solution further comprises a buffer.

19. The solution according to claim 14, wherein the concentration of the color change agent is from 1 micromolar to 1 millimolar, wherein the concentration of the antioxidant is from 1 micromolar to millimolar, or a combination thereof.

20. The solution according to claim 14, wherein the molar ratio of the color change agent to the antioxidant is from 0.00001 to 5000.

21. The solution according to claim 14, wherein the solution is stable for at least 3 weeks.

22. A kit for determining whether a wood has been treated with a metal, the kit comprising:
a compartment containing the solution of claim 14, and
an applicator for applying the solution to the wood.
